(19) United States
(12) Patent Application Publication
(10) Pub. No.: US 2026/0263790 A1
(43) Pub. Date: Sep. 10, 2026

(54) DEVICES AND RELATED ASPECTS FOR RECORDING AND STIMULATING GASTRIC ACTIVITY

(71) Applicants: THE JOHNS HOPKINS UNIVERSITY, Baltimore, MD (US); THE UNIVERSITY OF MELBOURNE, Parkville, VIC (AU)

(72) Inventors: Vivek GANESH, Baltimore, MD (US); Martin J. STEBBING, Victoria (AU); Pedro IRAZOQUI, Baltimore, MD (US)

(21) Appl. No.: 19/127,257

(22) PCT Filed: Nov. 17, 2023
(86) PCT No.: PCT/US2023/080192

§ 371 Date: May 5, 2025

Related U.S. Application Data
(60) Provisional application No. 63/384,126, filed on Nov. 17, 2022.

(51) Int. Cl. A61N 1/36; A61B 5/00; A61B 5/392; A61N 1/372

(52) U.S. Cl. CPC A61N 1/36007; A61B 5/392; A61B 5/4836; A61N 1/36135; A61N 1/37247

(57) ABSTRACT

Provided herein are implantable electroceutical devices. In some embodiments, an implantable electroceutical device includes a gastric motility sub assembly configured to record gastric myoelectric activity and gastric contraction activity in a subject when the implantable electroceutical device is operably implanted in the subject. In some embodiments, an implantable electroceutical device also includes a gastric electrical stimulation (GES) sub-assembly configured to electrically stimulate a nervous system of a subject when the implantable electroceutical device is operably implanted in the subject. Related implantable electroceutical devices, systems, methods, kits, and computer readable media are also provided.

### CROSS-REFERENCE TO RELATED APPLICATIONS

This application is the national stage entry of International Patent Application No. PCT/US2023/080192, filed on Nov. 17, 2023, and published as WO 2024/108073 A8 on May 23, 2024, which claims the benefit of U.S. Provisional Patent Application Ser. No. 63/384,126, filed on Nov. 17, 2022, which are hereby incorporated by reference herein in their entireties.

### BACKGROUND

Gastroparesis and functional dyspepsia (FD) are debilitating disorders characterized by a myriad of symptomatic conditions including postprandial discomfort, premature satiety to bloating, nausea, vomiting or regurgitation and epigastric pain. As a result, patients suffering from this condition report a significant reduction in quality of life. Together, they affect up to 10% of the population. Current treatment strategies target symptom management, including dietary modification, pro-kinetics and nutritional supplementation. Despite optimal medical therapy, patients with refractory gastroparesis require frequent hospitalizations which not only impairs quality of life but also causes significant financial hardship. For those patients with refractory gastroparesis, gastric electrical stimulation (GES) or gastric pacing are their primary options. GES is typically performed using electrodes and a pulse generator to electrically stimulate gastric tissues. Initial observations in dogs suggested that gastric electrical stimulation could ‘entrain’ the pacemaker activity of the stomach, modulate its frequency and increase gastric emptying. This effect was seen even after vagotomy, but only if the gastric pacemaker systems were intact and their rhythm was disrupted pharmacologically. It was suggested that such stimulation might increase the amplitude of peristaltic contractions if synchronized with slow waves and could resynchronize their activity that had become disorganized. This and other studies were used as a basis for a therapeutic device called the Enterra, an FDA-approved device that applies stimulation to the muscle in humans via an electrode pair implanted into the gastric musculature of the corpus near the greater curvatures. Unfortunately, this device does not have the ability to stimulate in synchrony with naturally occurring slow waves and cannot deliver the large currents and long stimulus pulses required to pace slow waves in human. An initial multicenter clinical trial in 38 patients demonstrated the safety of the approach and showed great promise in terms of efficacy, particularly for reduction in symptoms. Multiple studies since have shown a decrease in symptoms using this device, but few are placebo controlled and equally multiple studies have concluded that the device does not increase gastric emptying or significantly modify gastric motor patterns. A recent double blinded cross over trial concluded it could reduce vomiting scores in patients with refractory vomiting but did not improve quality of life. Several technical variables determine the effect of electrical stimulation on gut tissue. Preclinical studies on optimal stimulation parameters, electrode placement and target outcomes are all plagued by the fact that most were conducted in acute settings under general anesthesia. They share a major limitation where anesthesia can reduce gastric motility, with common anesthetics like isoflurane having been shown to reduce gastric motility by nearly 50% just 120 minutes after brief exposure.

The biological and mechanistic basis for the utility of the implant, and the stimulus modality it uses, was not properly established before moving forward to clinical trial and use in humans. The inability of the device to increase gastric emptying in humans appears predictable based on both current knowledge of the biology of the system and on preclinical studies in animals-it is not due to a lack of utility or applicability of the preclinical testing process. In the absence of any effect of the device on the function of the stomach, the biological basis for any reduction in symptoms and vomiting is not clear, but a strong placebo effect that is common in device implantation studies may contribute. Therefore, before application of this device can be improved, or any alternative therapy trialed, a full understanding of the biology, the practical considerations and the safety of the approach are desirable.

Accordingly, there is a need for additional techniques for GES, which can be achieved through the use of a single system capable of not only administering GES therapy, but also recording resulting gut tissue response.

### SUMMARY

In some aspects, this disclosure provides a fully implantable system for wireless recording of gastric myoelectrical activity via electrogastrography (EGG) and gastric contraction activity via a force transducer (strain gauge), as a tool to assess overall gastric motility state, in a subjects. In some embodiments, the system is capable of administering GES in parallel with recording motility. These and other attributes of the present disclosure will be apparent upon a complete review of the specification, including the accompanying figures.

In one aspect, the present disclosure provides an implantable electroceutical device to treat and monitor a gastric disorder in a subject. The implantable electroceutical device includes a gastric motility sub-assembly configured to record gastric myoelectric activity and gastric contraction activity in the subject when the implantable electroceutical device is operably implanted in the subject, and a gastric electrical stimulation (GES) sub-assembly configured to electrically stimulate a nervous system of the subject when the implantable electroceutical device is operably implanted in the subject. The implantable electroceutical device also includes a controller operably connected, or connectable, to the gastric motility sub-assembly and to the GES sub-assembly, which controller comprises, or is capable of accessing, computer readable media comprising non-transitory computer-executable instructions, which when executed by at least one electronic processor, perform at least: effecting recording of the gastric myoelectric activity and the gastric contraction activity in the subject using the gastric motility sub-assembly when the implantable electroceutical device is operably implanted in the subject; and effecting electrically stimulating the nervous system of the subject using the GES sub-assembly when the implantable electroceutical device is operably implanted in the subject. In addition, the implantable electroceutical device also includes a power source operably connected, or connectable, to the controller, the gastric motility sub-assembly, and/or the GES sub-assembly.

In some embodiments, the gastric motility sub-assembly comprises an electrogastrography (EEG) apparatus configured to record the gastric myoelectric activity in the subject when the implantable electroceutical device is operably implanted in the subject. In some embodiments, the gastric motility sub-assembly comprises a strain gauge force transducer configured to record the gastric contraction activity in the subject when the implantable electroceutical device is operably implanted in the subject. In some embodiments, the gastric motility sub-assembly configured to record physiologically relevant gastric motility patterns and changes in the subject when the implantable electroceutical device is operably implanted in the subject. In some embodiments, the gastric motility sub-assembly is configured to substantially continuously record the gastric myoelectric activity and the gastric contraction activity in the subject when the implantable electroceutical device is operably implanted in the subject.

In some embodiments, the non-transitory computer-executable instructions, when executed by the electronic processor, are configured to effect recording of the gastric myoelectric activity and the gastric contraction activity in the subject and to effect electrically stimulating the nervous system of the subject substantially synchronous with one another. In some embodiments, the non-transitory computer-executable instructions, when executed by the electronic processor, are configured to use continuous wavelet transforms to analyze frequency and amplitude changes in captured data to inform potential therapies for the subject when the implantable electroceutical device is operably implanted in the subject. In some embodiments, the non-transitory computer-executable instructions, when executed by the electronic processor, are configured to effect selective stimulation of enteric neurons of the subject in synchronization with recording gastric slow waves in the subject when the implantable electroceutical device is operably implanted in the subject. In some embodiments, the controller is configured for bi-directional wireless communication with a data acquisition computer readable media that is external to the subject.

In some embodiments, the gastric disorder comprises gastroparesis and/or functional dyspepsia (FD). In some embodiments, the implantable electroceutical device is configured for chronic implantation in the subject.

In some embodiments, the implantable electroceutical device is configured to administer closed-loop therapy to the subject when the implantable electroceutical device is operably implanted in the subject, the closed-loop therapy increases gastric accommodation, stimulates physiological gastric emptying, and/or stimulates pyloric opening in the subject when the implantable electroceutical device is operably implanted in the subject. In some embodiments, the implantable electroceutical device further comprises at least one external drive coil, at least one external base station, and/or at least one external computer operably connected, or connectable, to the implantable electroceutical device when the implantable electroceutical device is operably implanted in the subject. In some embodiments, the implantable electroceutical device further comprises one or more electrodes positioned within sensory communication of a stomach of the subject and operably connected to the implantable electroceutical device when the implantable electroceutical device is operably implanted in the subject.

In some embodiments, a system comprises the implantable electroceutical device. In some embodiments, a kit comprises the implantable electroceutical device.

In another aspect, the present disclosure provides a method of treating and monitoring a gastric disorder in a subject. The method includes recording gastric myoelectric activity and gastric contraction activity in the subject having the gastric disorder using an implantable electroceutical device that is operably implanted in the subject. The method also includes electrically stimulating a nervous system of the subject having the gastric disorder using the implantable electroceutical device that is operably implanted in the subject. In some embodiments, the method further includes operably implanting the implantable electroceutical device in the subject. In some embodiments, the implantable electroceutical device comprises: a gastric motility sub-assembly configured to record gastric myoelectric activity and gastric contraction activity in the subject; a gastric electrical stimulation (GES) sub-assembly configured to electrically stimulate the nervous system of the subject; a controller operably connected, or connectable, to the gastric motility sub-assembly and to the GES sub-assembly, which controller comprises, or is capable of accessing, computer readable media comprising non-transitory computer-executable instructions, which when executed by at least one electronic processor, perform at least: effecting the recording of the gastric myoelectric activity and the gastric contraction activity in the subject using the gastric motility sub-assembly; and effecting the electrically stimulating of the nervous system of the subject using the GES sub-assembly; and, a power source operably connected, or connectable, to the controller, the gastric motility sub-assembly, and/or the GES sub-assembly.

In another aspect, the present disclosure provides a computer readable media comprising non-transitory computer-executable instructions which, when executed by at least one electronic processor perform at least: effecting recording of gastric myoelectric activity and gastric contraction activity in a subject using a gastric motility sub-assembly of an implantable electroceutical device that is operably implanted in the subject; and effecting electrically stimulating a nervous system of the subject using a gastric electrical stimulation (GES) sub-assembly of the implantable electroceutical device that is operably implanted in the subject.

### BRIEF DESCRIPTION OF DRAWINGS

FIG. 1 is a flow chart that schematically shows exemplary method steps of treating and monitoring a gastric disorder in a subject according to some aspects disclosed herein.

FIG. 2 is a schematic diagram of an exemplary system suitable for use with certain aspects disclosed herein.

FIG. 3. A; Actual device, packaged in silicone, with two patch electrode leads and one strain gauge. B; Micro-CT image of device in A, showing internal circuitry, secondary coil, and battery. C; Block diagram illustrating working principles of the implantable system.

FIG. 4. A; Half bridge amplifier circuit with dynamic calibration, using a dual element strain gauge. B; Flexible PCB with strain gauge lead. C; Gastric contraction activity, wirelessly recorded from corpus, in a freely behaving rat after applying a 0.006 s moving window average filter.

FIG. 5. A cross-sectional view of packaging the Gastronode. A; Motherboard (longest left-most vertical rectangular strip with thicker parallel cross-hatching lines facing upward to the right) connected with strain gauge flex PCB (longest right-most vertical rectangular strip with larger speckled fill) with gold pins (shorter rectangles with smaller specked fill) and with feedthrough board (shown at the top, oriented horizonally in contact with both the motherboard and the flex PCB). B; First layer of parylene (layer shown over both the motherboard and the flex PCB) applied. C; Electrode leads attached to feedthrough board, insulated except for the junction, and second layer of parylene (layer shown over the first layer of parylene) applied. D; Gastronode encased in silicone (outermost hashed line rectangular box).

FIG. 6. Illustration of the process of implanting the Gastronode in a rat. A; The procedure begins with the animal prone, and the Gastronode implanted subcutaneously. B; Leads are routed along the flank using a trocar, to the abdomen. C; Electrodes are sutured to the stomach serosa.

FIG. 7. Timeline of chronic data collection. Time points are in relation to the start of fasting.

FIG. 8. A; Percent change in cycles per minute in acute rats from baseline versus time post-injection of Metoclopramide (experimental) or saline (control), showing the mean value, with error bars representing the SEM. Asterisks indicate statistically significant differences from baseline between both groups. * corresponds to p<0.05, ** corresponds to p<0.01. Minute 0 corresponds to baseline. Peak change occurs 35-45 mins post injection in experimental group. B; Example 3-minute raw EGG from a control animal, showing 4.5 cpm at baseline and 4.5 cpm 45-min post saline injection. C; Example 3-minute raw EGG from an experimental animal, showing 4.2 cpm at baseline and 5cpm 45-min post metoclopramide injection.

FIG. 9. A; Baseline chronic EGG (left) and strain gauge (right) showing gastric myoelectric and contraction activity for 30 minutes preprandial after fasting for 18 h. B; 10-min feeding and postprandial chronic EGG (left) and strain gauge (right) showing gastric myoelectric and contraction activity for 75 minutes. C; Example 8-minute comparison of strain gauge (upper trace) and EGG (lower trace) at the beginning of the postprandial phase. The region in between the black lines, in B and C, is the region of interest.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

In some embodiments: As used herein, the term “in some embodiments” refers to embodiments of all aspects of the disclosure, unless the context clearly indicates otherwise.

Subject: As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, terms “individual” or “patient” are used and are intended to be interchangeable with “subject.”

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

System: As used herein, “system” in the context of analytical instrumentation refers a group of objects and/or devices that form a network for performing a desired objective.

### DETAILED DESCRIPTION

In some aspects, the present disclosure provides implantable electroceutical devices and related methods of treating and monitoring a gastric disorder in a subject. Some embodiments include a closed loop, chronically implantable electroceutical device capable of continuously recording gastric motility and administering synchronous GES, that forms a needed foundation for neuromodulation protocols that can correct shortcomings in earlier generation bioelectronic attempts to ameliorate and monitor gastric disorders. In some embodiments, the systems disclosed herein capture gastric serosal myoelectric activity using electrogastrography, as well as gastric contraction activity using strain gauge force transducers. In some embodiments, the devices capture physiologically relevant gastric motility patterns and changes, safely and effectively. In some embodiments, the frameworks of the present disclosure are built on continuous wavelet transforms to analyze frequency and amplitude changes in captured data to inform potential therapies. In some embodiments, the devices have the ability to selectively stimulate enteric neurons synchronous with gastric slow waves, resulting in a relaxation of the pyloric sphincter muscle, in a closed loop fashion. In some embodiments, the closed loop therapies are aimed at increasing gastric accommodation, stimulating physiological gastric emptying and/or pyloric opening with physiologically appropriate timing and extent. These and other attributes of the present disclosure will be apparent upon a complete review of the specification, including the accompanying figures.

To illustrate, the present disclosure provides an implantable electroceutical device to treat and monitor a gastric disorder in a subject. The implantable electroceutical device includes a gastric motility sub-assembly configured to record gastric myoelectric activity and gastric contraction activity in the subject when the implantable electroceutical device is operably implanted in the subject, and a gastric electrical stimulation (GES) sub-assembly configured to electrically stimulate a nervous system of the subject when the implantable electroceutical device is operably implanted in the subject. The implantable electroceutical device also includes a controller operably connected, or connectable, to the gastric motility sub-assembly and to the GES sub-assembly, which controller comprises, or is capable of accessing, computer readable media comprising non-transitory computer-executable instructions, which when executed by at least one electronic processor, perform at least: effecting recording of the gastric myoelectric activity and the gastric contraction activity in the subject using the gastric motility sub-assembly when the implantable electroceutical device is operably implanted in the subject; and effecting electrically stimulating the nervous system of the subject using the GES sub-assembly when the implantable electroceutical device is operably implanted in the subject. In addition, the implantable electroceutical device also includes a power source operably connected, or connectable, to the controller, the gastric motility sub-assembly, and/or the GES sub-assembly.

In some embodiments, the gastric motility sub-assembly comprises an electrogastrography (EEG) apparatus configured to record the gastric myoelectric activity in the subject when the implantable electroceutical device is operably implanted in the subject. In some embodiments, the gastric motility sub-assembly comprises a strain gauge force transducer configured to record the gastric contraction activity in the subject when the implantable electroceutical device is operably implanted in the subject. In some embodiments, the gastric motility sub-assembly configured to record physiologically relevant gastric motility patterns and changes in the subject when the implantable electroceutical device is operably implanted in the subject. In some embodiments, the gastric motility sub-assembly is configured to substantially continuously record the gastric myoelectric activity and the gastric contraction activity in the subject when the implantable electroceutical device is operably implanted in the subject.

In some embodiments, the non-transitory computer-executable instructions, when executed by the electronic processor, are configured to effect recording of the gastric myoelectric activity and the gastric contraction activity in the subject and to effect electrically stimulating the nervous system of the subject substantially synchronous with one another. In some embodiments, the non-transitory computer-executable instructions, when executed by the electronic processor, are configured to use continuous wavelet transforms to analyze frequency and amplitude changes in captured data to inform potential therapies for the subject when the implantable electroceutical device is operably implanted in the subject. In some embodiments, the non-transitory computer-executable instructions, when executed by the electronic processor, are configured to effect selective stimulation of enteric neurons of the subject in synchronization with recording gastric slow waves in the subject when the implantable electroceutical device is operably implanted in the subject. In some embodiments, the controller is configured for bi-directional wireless communication with a data acquisition computer readable media that is external to the subject.

In some embodiments, the gastric disorder comprises gastroparesis and/or functional dyspepsia (FD). In some embodiments, the implantable electroceutical device is configured for chronic implantation in the subject.

In some embodiments, the implantable electroceutical device is configured to administer closed-loop therapy to the subject when the implantable electroceutical device is operably implanted in the subject, the closed-loop therapy increases gastric accommodation, stimulates physiological gastric emptying, and/or stimulates pyloric opening in the subject when the implantable electroceutical device is operably implanted in the subject. In some embodiments, the implantable electroceutical device further comprises at least one external drive coil, at least one external base station, and/or at least one external computer operably connected, or connectable, to the implantable electroceutical device when the implantable electroceutical device is operably implanted in the subject. In some embodiments, the implantable electroceutical device further comprises one or more electrodes positioned within sensory communication of a stomach of the subject and operably connected to the implantable electroceutical device when the implantable electroceutical device is operably implanted in the subject.

In some embodiments, a system comprises the implantable electroceutical device. Exemplar systems are described further herein. In some embodiments, a kit comprises the implantable electroceutical device.

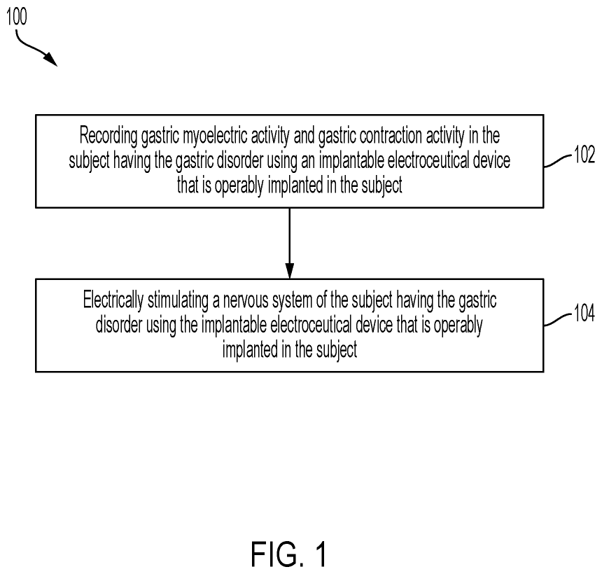

By way of further example, FIG. 1 is a flow chart that schematically shows exemplary method steps of treating and monitoring a gastric disorder (e.g., gastroparesis, functional dyspepsia (FD), etc.) in a subject according to some aspects disclosed herein. As shown, method 100 includes recording gastric myoelectric activity and gastric contraction activity in the subject having the gastric disorder using an implantable electroceutical device that is operably implanted in the subject (step 102). Method 100 also includes electrically stimulating a nervous system of the subject having the gastric disorder using the implantable electroceutical device that is operably implanted in the subject (step 104).

In some embodiments, the method further includes operably implanting the implantable electroceutical device in the subject. In some embodiments, the implantable electroceutical device comprises: a gastric motility sub-assembly configured to record gastric myoelectric activity and gastric contraction activity in the subject; a gastric electrical stimulation (GES) sub-assembly configured to electrically stimulate the nervous system of the subject; a controller operably connected, or connectable, to the gastric motility sub-assembly and to the GES sub-assembly, which controller comprises, or is capable of accessing, computer readable media comprising non-transitory computer-executable instructions, which when executed by at least one electronic processor, perform at least: effecting the recording of the gastric myoelectric activity and the gastric contraction activity in the subject using the gastric motility sub-assembly; and effecting the electrically stimulating of the nervous system of the subject using the GES sub-assembly; and, a power source operably connected, or connectable, to the controller, the gastric motility sub-assembly, and/or the GES sub-assembly.

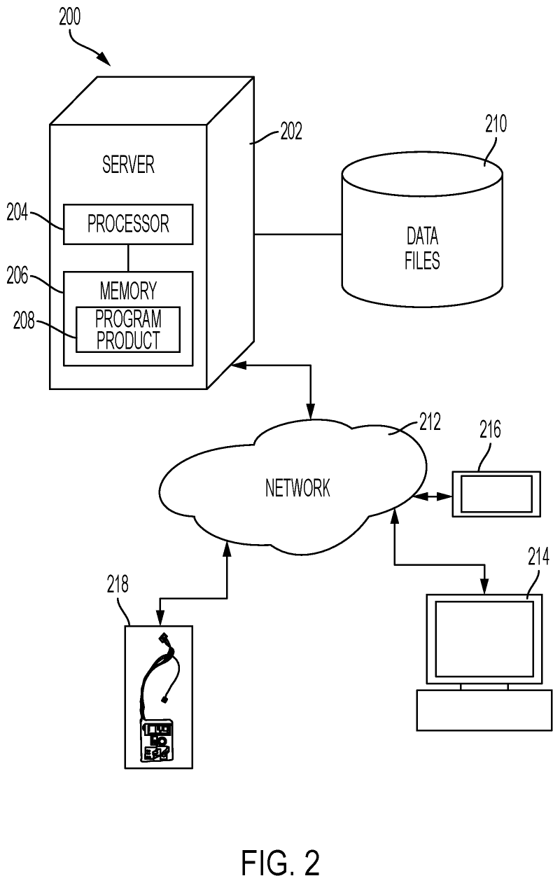

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server or configured as a base station in some embodiments), which includes processor 204 and memory, storage device, or memory component 206, and one or more other communication devices 214, 216, (e.g., base stations, client-side computer terminals, telephones, tablets, laptops, other mobile devices, etc. (e.g., for receiving data sets or results, etc.) in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism.

System 200 also includes program product 208 (e.g., for performing the methods as described herein) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to gastric myoelectric activity and gastric contraction activity data, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a base station, laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 208 includes non-transitory computer-executable instructions which, when executed by electronic processor 204, perform at least: effecting recording of gastric myoelectric activity and gastric contraction activity in a subject using a gastric motility sub-assembly of an implantable electroceutical device that is operably implanted in the subject; and effecting electrically stimulating a nervous system of the subject using a gastric electrical stimulation (GES) sub-assembly of the implantable electroceutical device that is operably implanted in the subject

In some embodiments, the methods of the present disclosure are performed using implantable electroceutical device 218 or another device disclosed herein to treat and monitor a gastric disorder in a subject when the implantable electroceutical device is operably implanted in the subject. Additional details are provided in Appendices A and B, which are incorporated by reference in their entirety.

### Example: Chronically Implantable Wireless Electroceutical Device for Recording

### Gastric Motility in Freely Behaving Rats

### 1. Introduction

Gastroparesis and functional dyspepsia are debilitating stomach disorders. Modulating gastric function is an important target function for alternative therapies like gastric electrical stimulation (GES). We present the development and in vivo application of new, chronically implantable wireless system capable of continuously recording gastric motility, that will form the needed foundation for neuromodulation protocols that can correct shortcomings in past, first-generation bioelectronic attempts to ameliorate and monitor gastric disorders.

### 2. Materials and Methods

### 2.1 Implant System Overview

We have previously developed an implantable electroceutical, the Bionode, capable of recording biopotential signals and electrically stimulating the nervous system primarily in rodents. An external base station facilitates bi-directional Wi-Fi based communication between a PC-based data acquisition software and the device. The Gastronode was built on these foundations but developed and modified for chronic recording and stimulation of gastric activity. It consists of a NRF52832 microcontroller with an embedded 2.4 GHz radio, 12-bit Analog to Digital Converter (ADC) and a total sample rate up to 50 kHz, programmable constant current stimulation circuit allowing biphasic stimulation up to +2.25 mA, voltage rails up to +7.5V, and a custom analog front end (AFE) capable of filtering, amplifying, and conditioning up to 6 biopotential signals. The device is wirelessly powered at 13.56 MHZ ISM band, with onboard powering circuitry designed to provide an average power of 31 mW with a peak power consumption of 54 mW when stimulating. The device uses a two-coil system, designed using equations for coupling coefficient optimization. The device uses a 11.58 mm outer diameter, 6-turn, concentric, secondary coil made from 22-gauge wire hand wrapped around a K1 ferrite material core (relative permeability 11r=80 at 13.56 MHz) to harvest energy from circulating magnetic fields from a primary pancake coil with 3 turns, an outer diameter of 12.5 cm, with 11 mm interwinding spacing, and wound using 10-gauge wire. We further optimized for number of turns and wire-gauge choice by iterative experimentation and measurements of S21 characteristics using a vector network analyzer. The coupled AC power undergoes full wave rectification to DC power, ultimately powering low dropout regulators (LDR's) supplying 7.4V, 1.8V and 0.9V for all digital, recording and stimulating circuitry. The external base station drives the 13.56 MHz wireless source signal at a peak power of 17.8 dBm. An external power amplifier (ZHL-1-2 W-S+, MiniCircuits, NY, USA) is connected to the base station to provide peak power up to 40 dBm or 10 Watts, which is sufficient to power the device implanted subcutaneously, at distances up to 8 cm when using the primary coil. The device can recharge an onboard 130 mAh 3.7 V Lithium-Ion battery, used as the primary power source during all recording sessions and whose voltage level is continuously monitored to allow scheduled recharging sessions.

### 2.1.1 Strain Gauge

While the crude diagnostic tools commonly used in clinics may not always show decreased gastric emptying, it is widely accepted that the underlying cause of the symptoms in gastroparesis/FD is a defect in gastric and or pyloric motor control with or without sensitization to gastric stimuli. Lu et al. (“Automatic assessment of human gastric motility and emptying from dynamic 3D magnetic resonance imaging,” Neurogastroenterol Motil. 2022 January; 34 (1): e14239) developed new MRI methods to study gastric motility in detail using long imaging sessions with temporal resolution appropriate for measuring gastric peristalsis, confirming the motor component of these disorders. This and other studies suggest that a range of gastric movement disorders can be distinguished in patients with the same symptomatology and clinical diagnosis. Interestingly they report that there is usually no obvious change in the frequency of the rhythmic contractions (or by deduction the gastric slow waves) measured at any one point in the stomach in most patients. They confirm that decreased accommodation occurs, as reported in other studies, which presumably contributes to symptoms of premature satiety. While the types of defects in gastric motility can vary greatly between patients, a unifying feature is a lack of coordination between contractions and electrical activity in different gastric regions and/or between antral contractions and pyloric opening. In order to study the temporal relationship between contractions and electrical activity in different gastric regions, we opted to use implantable strain gauge force transducers. These were developed in collaboration with Clunbury Scientific, MI, USA and fabricated using the step-by-step documentation based on the original designs of Bass and colleagues (Holmes et al., “Fabrication and implantation of miniature dual element strain gages for measuring in vivo gastrointestinal contractions in rodents,” J Vis Exp. 2014; (91): 51739) with some modifications. The use of force transducers typically involve a wheatstone bridge circuit. However, major drawbacks of conventional wheatstone bridges include superposition of normal strain and bending strain, and poor tolerance to temperature effects and the lack of a compensation mechanism to account for this. There are four different types of strain, namely axial, bending, shear and torsional strain. Given the slow wave behavior and retrograde propagation of contractions, we hypothesized that bending strain would be the most predominant, along the circumferential smooth muscles. In order to compensate for temperature effects and also separate bending strain from normal strain, a half-bridge configuration using dual element strain gauges was used. By making both gauges active in the half-bridge configuration, the bridge's sensitivity to strain was doubled since one gauge measures tensile strain, while the other gauge measures compressive strain.

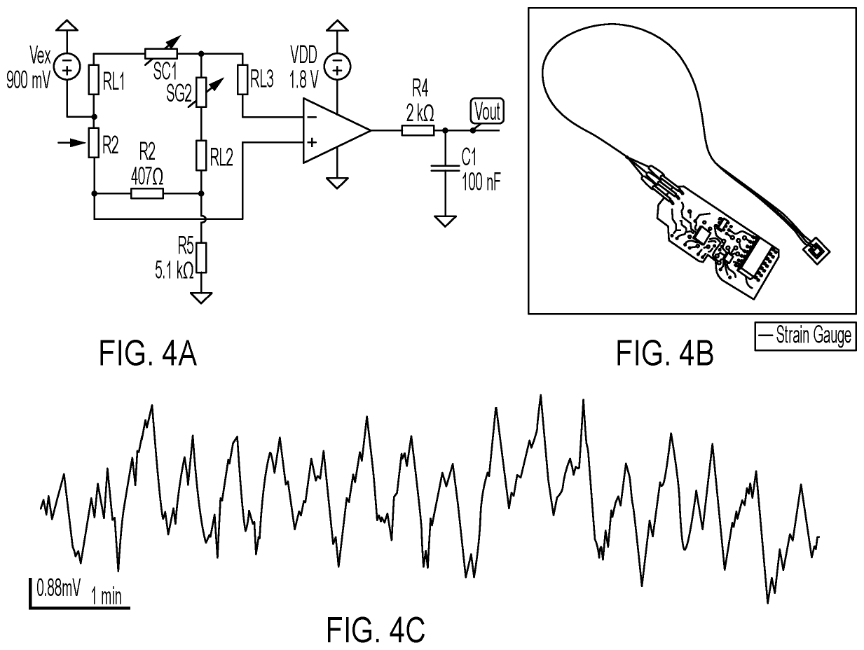

To complete the circuit, a 1.8V instrumentation amplifier (INA) was used with an output RC 795 Hz low-pass filter prior to feeding into the ADC. A 0.9V excitation voltage is used to allow proper common mode voltage setup for the INA. Given the nominal resistance of each element of the dual element strain gauge was on average 407 (nominal resistance of 350 Ohms+leadwire resistance), equivalent resistor values were used to balance the bridge. In practice, lead wires connecting the strain gauge to the bridge will add some measurable resistance that could cause a significant lack of symmetry and an unbalanced condition. In this case, the lead wire resistance was at least 50 Ohm. This could lead to a non-zero output voltage at the high impedance inputs to the INA. After applying gain, this could limit the available measurement range for the downstream ADC. Additional temperature changes can cause significant differences. To compensate for this and ensure symmetry, a three-wire configuration with the dual element gauges was used. In this configuration, leadwire RL1 and SG1 comprise one arm of the bridge, and RL2 and SG2 comprise the adjacent arm (FIG. 4). The negative output bridge corner is electrically moved to the end of RL3. In this way, the bridge remains balanced regardless of temperature changes, so long as the lead wires are at the same respective temperature. RL3 is not in series with any of the bridge arms but is in series with the high impedance INA input, and therefore it has no effect on bridge balance or temperature stability.

The need for calibration arises quite frequently in strain gauge instrumentation. Particularly when used to measure serosal gastric contractions, there may be a slow drift from baseline in strain measurements over time. For chronic recordings of motility, it is necessary to compensate for any periodic drift to ensure accuracy and linearity of the instrumentation itself. Conventionally, there are two methods for calibration-direct and indirect. Direct calibration involves the use of a precise mechanical input to the gauge and subsequent comparison with the output for verification or adjustment purposes. This technique typically results in a calibration curve that is then later referenced at the conclusion of any experiments. Indirect calibration involves the use of a shunt resistor across one of the arms of the wheatstone bridge to simulate the action of a strain gauge. This is the most commonly used and widely accepted technique in the field. When properly done, the output of the instrumentation is once again compared to a predetermined value and if the output deviates from this correct value, then the error is due to the instrumentation. This would then entail calibration or repairs to the instrumentation. An important distinction here is that calibration in the conventional sense of strain gauge instrumentation is meant to account for deviations in the instrumentation, and not for deviations in the strain gauge itself.

We developed a way to compensate for any non-linear behavior in the strain gauge transducer itself to prevent drift and subsequent reduction in resolution. The equation for a half bridge circuit can be written as

$${V}_{o} = [ \frac{{SG}_{1} / {R}_{2}}{{SG}_{1} / {R}_{2} + 1} - \frac{{SG}_{2} / {R}_{3}}{{SG}_{2} / {R}_{3} + 1} ] * {V}_{ex} \qquad ( 1 )$$

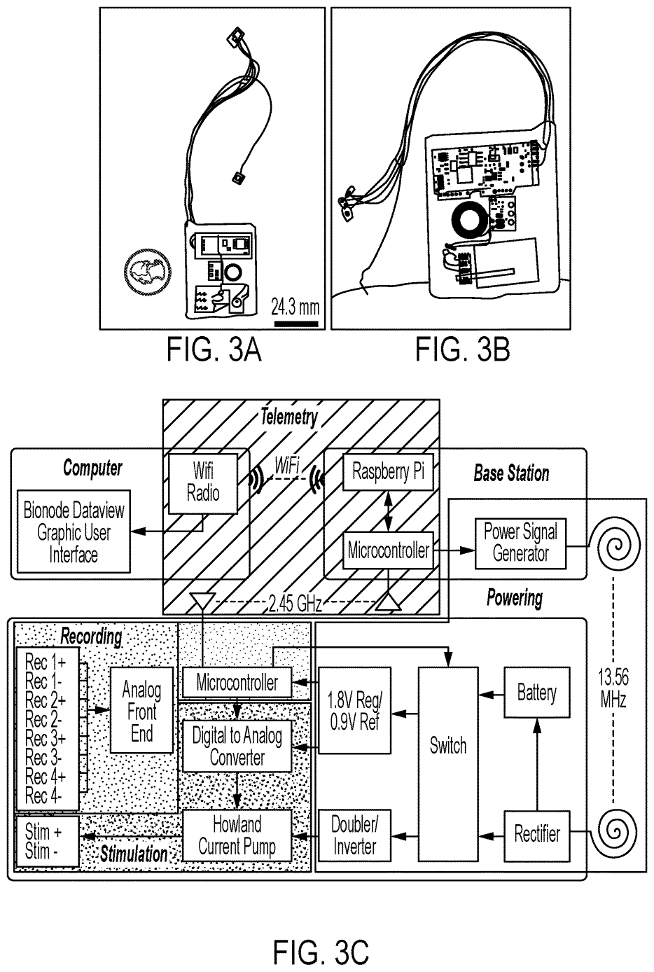

It is evident from (1) that the output depends on the resistance ratios SG₁/R₂ and SG₂/R₃, rather than on individual resistances. When these are equal, the bridge is fully balanced, and the output voltage will be zero. This would translate to Vₒᵤₜ=0.9V (See FIG. 2). In order to dynamically calibrate and re-center any offset voltages introduced from non-linearities in the wheatstone bridge as it is implanted on the serosal surface of the stomach, a 256-tap 1 k-Ohm digital potentiometer was used to allow on-the-fly calibration. Increasing or decreasing this resistance until the bridge is balanced (zero measured voltage at zero strain), would allow the user to compensate for any drift in Vₒᵤₜ and therefor ensure a full measurement range. Settings for this user-configurable calibration are sent via RF signals to the device (FIG. 3).

### 2.1.2 EGG

Almost all current and proposed treatments for gastroparesis that target mechanism rather than symptoms, are designed to increase gastric emptying. A consistent finding of multiple studies is that the rate of gastric emptying does not correlate with symptoms in these patients. Despite this, gastric emptying is clearly and consistently delayed in a subset of patients with gastroparesis/FD symptoms and inconsistently delayed in another cohort. Even if delayed gastric emptying is not the direct cause of symptoms, then a treatment that could stimulate physiological gastric emptying with appropriate timing and extent, would be predicted to reduce symptoms of bloating and premature satiety, based on the reported efficacy of pyloroplasty and pyloromyotomy. Recording gastric myoelectric activity in addition to contractile activity can serve as an effective tool to enable studies aimed at this goal. To record gastric myoelectric activity, we used the EGG technique which can detect gastric slow waves at predominant frequencies. We designed custom in-house bipolar patch electrodes using Platinum90/Iridium 10 (90/10 Pt/Ir) wire (Fort Wayne Metals, IN, USA,) and 0.08″ silicone sheets and silicone tubing (A-M systems, WA, USA). The impedance between the electrode contacts in physiological saline was measured as 0.4-0.6 kD using a Gamry Ref600+potentiostat/galvanostat/ZRA (Gamry Instruments Inc., PA, USA). The electrodes measured 7.17 mm wide and 4.2 mm long, with a 15 cm lead length. Each contact plate measured 1 mm wide and 2 mm long, with a separation of 2.76 mm between both contact plates. In rats, slow waves typically occur at a frequency of 3-5 cycles per minute (cpm) or 0.05-0.08 Hz. Following simulations, the AFE was modified to achieve a bandpass of 0.046 Hz-142 Hz with a peak gain of 60 dB.

### 2.1.3 Packaging

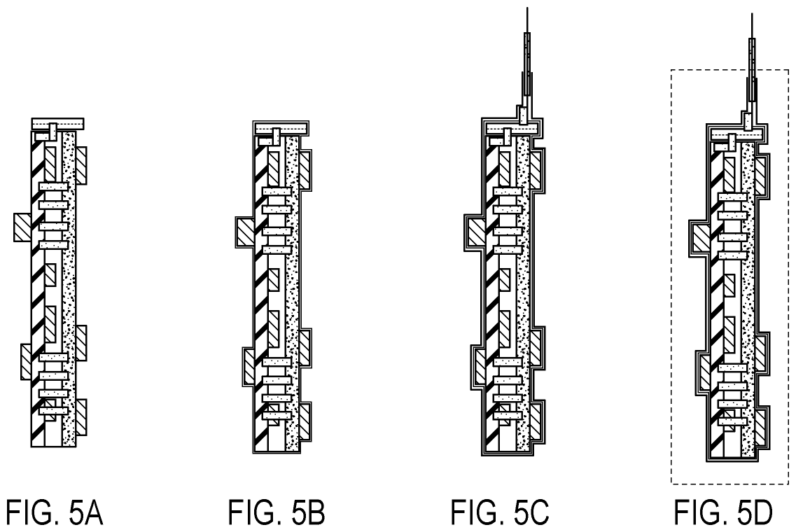

A multi-step process, shown in FIG. 5, was developed to assemble, seal and package the Gastronode to ensure hermeticity prior to implant. In the first step, a 15-um layer of Parylene C was deposited over the Gastronode assembly, without leads attached to the feedthrough board (FIG. 5A, B). After the first layer, the patch electrode and strain gauge leads were electrically connected to the feedthrough board before applying a second 15-um layer parylene deposition with the electrodes shielded (to avoid insulating the conductive area of the patch electrode) (FIG. 5C). 9 mL each of Ecoflex 00-30 Part A and Part B silicone (Smooth-On Inc., PA, USA) was mixed and pressurized down to vacuum (760 bar) for 5 mins to remove any air bubbles. This mixture was brought back to atmospheric pressure, poured into a custom 3D printed mold containing the Gastronode (FIG. 5D), and the entire assembly was then pressurized down to vacuum for a second time to remove air bubbles, before being brought back to atmospheric pressure and allowed to cure for 4 hours.

### 2.2 Animal Protocol and Experiment Design

Ten rats (Long Evans, male and female, 250-400 g, Envigo, Indianapolis, IN) were included in the study according to procedures approved by the Johns Hopkins Animal Care and Use Committee. For the acute experiments, 3 male and 3 female rats were used. In the experimental group (n=3) a comparison of EGG recordings at baseline and following a bolus injection of 10 mg/kg subcutaneous (s.c.) Metoclopramide (Hospira worldwide, IL, USA) was carried out. Metoclopramide was chosen as a pro-motility agent since it is known to increase the rate of gastric emptying. In the control group (n=3) a bolus injection of 10 mg/kg s.c. saline was administered instead. In the chronic setting, 2 male and 2 female rats were implanted for 14 days. In each rat, strain gauge and EGG recordings were compared at baseline (no fasting) and pre and postprandial after fasting for 18 hours. Animals were housed individually with ad-lib access to food and water, except when fasted. The environment was maintained on a 12: 12 h light-dark cycle (lights on at 6 am and lights on at 6 μm).

### 2.3 Animal Preparation and Surgical Procedures

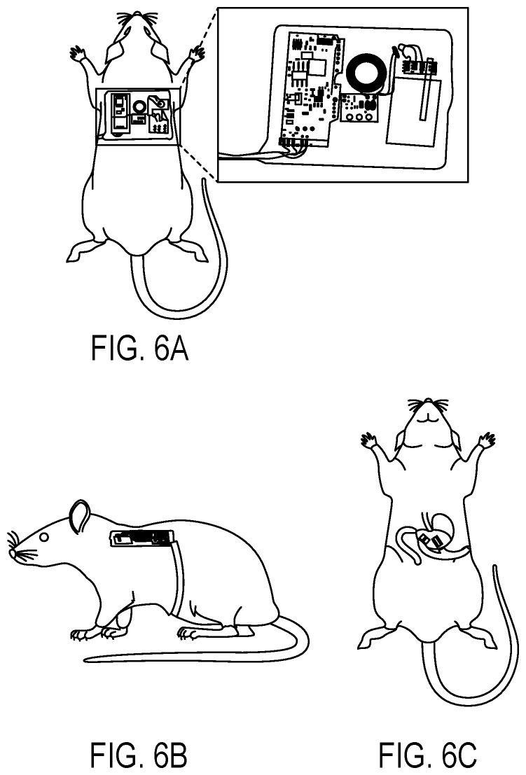

For the acute experiments, animals were initially anesthetized with 4% isoflurane in oxygen at a flow rate of 2 L/min. The animal was placed in the supine position on a thermostatically controlled electric heating pad which monitored body temperature via anal probe. Heart rate and blood oxygenation were monitored using a NONIN 8600V Pulse Oximeter on the right hindpaw. Adequate anesthesia was confirmed before and during the surgery at regular intervals via the absence of a response to toe-pinch. After shaving the region, a 5 cm abdominal incision was made, just below the sternum. Anesthesia was reduced to 1.5%-2.0% isoflurane mixed with oxygen at a flow rate of 2 L/min throughout the surgery. After a midline laparotomy, the strain gauge transducer was sutured on 4 corners, using 6-0 silk (Med-Vet International, IL, USA) to the serosal surface of the stomach, in the corpus, with the strain gauge sensing axis oriented parallel to the circular muscle. The EGG patch electrode was placed on the antrum, 2 cm proximal to the pyloric sphincter, with a line through the two electrode contacts longitudinally aligned and sutured on 4 corners to the serosal surface of the stomach using 6-0 silk. Physiological saline solution was infused into the peritoneal space (2 mL/h i.p.) manually. The electrode leads were then connected to the Gastronode externally. The peritoneum was sutured closed using running 4-0 PGA sutures (Med-Vet International, IL, USA), and similarly, the abdominal muscle was also sutured closed. For the chronic experiments, surgery and anesthesia was similar, except that the animal was placed in the prone position initially and a s.c. bolus injection of 0.04 mL of 10 mg/ml Vetorphic (VetOne, Paris, FR) was administered before the first incision and every 3-4 hours until the surgery was completed. A 5 cm lateral incision was made above the shoulders and a pocket was excavated s.c. above the scapula to house the flexible Gastronode package. The Gastronode was sutured in place on four corners using 4-0 silk sutures and the lateral incision was closed using 4-0 PGA (FIG. 6A). Electrode leads were routed via a trocar around the left side of the abdomen, into the peritoneum (FIG. 6B). Thereafter, the strain gauge and patch electrode were sutured to the serosal surface of the corpus and antrum as for acute experiments via a midline laparotomy (FIG. 6C). Running 4-0 PGA sutures were used to close the peritoneum and abdominal muscles independently. Finally, the skin was closed using wound staples (Autoclip System, Fine Science Tools, CA, USA) to ensure patent wound closure during healing. Immediately following surgery, animals received a bolus injection of 0.1 ml per 200 g Ethiqa XR Buprenorphine (Fidelis Animal Health Inc., NJ, USA) s.c. and a second dose after 72 hours, a bolus injection of 0.05 ml Cefazolin (Hikma Pharmaceuticals, NJ, USA) s.c. and every 9-12 hours for a period of 72 hours. The staples were removed 10 days post-operatively, when the wound was sufficiently healed. At the conclusion of acute experiments and after 14 days of chronic implant, the animal was euthanized with 0.9 ml of 50 mg/ml pentobarbitone i.p. (Euthasol, Virbac Corporation, TX, USA).

TABLE 1
Overview of animal subgroups used

| Type | Experiment | Number |
|---|---|---|
| Acute | Metoclopramide | n = 3 |
| | Control | n = 3 |
| Chronic | Baseline | n = 4 |
| | Preprandial + | |
| | Postprandial | |
| | after fasting | |

### 2.4 Gastric Activity Measurements

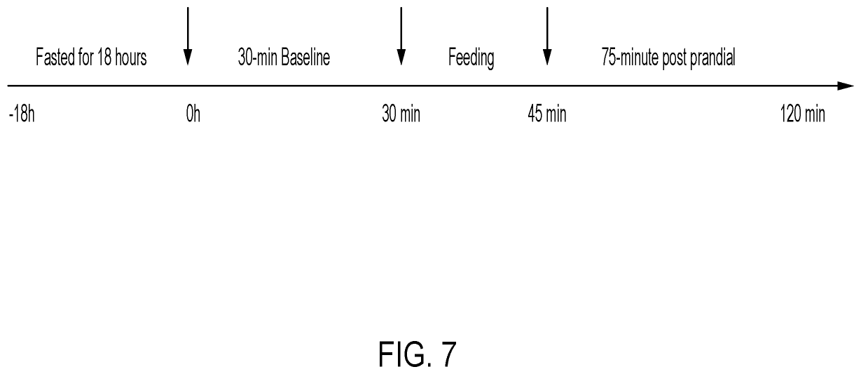

The Gastronode was powered on battery for the duration of all recordings. For the acute experiments, baseline data was recorded for 30 minutes, after which a bolus injection of 10 mg/kg s.c. Metoclopramide was administered, and data was collected for an additional 60 minutes. For the chronic experiments, following at least 7 days of post-op recovery, 60-minutes of baseline data was collected from each implanted animal on day 8 and 9 in the afternoon. Following fasting for 18 hours, on day 10, and day 13, preprandial data was collected for 30 minutes and then animals were given a fixed quantity of 10 g of DietGel (DietGel Recovery, ClearH2O, ME, USA), which was consumed on average within 14±5 minutes. Postprandial data was then collected immediately after the meal for 75 minutes This timeline is shown below in FIG. 7. Following a fasting experiment, animals had ad lib access to food and water.

### 2.5 Wavelet Analysis

The noise inherent in high resolution gastric serosal recordings has previously been classified as a combination of baseline drift and high frequency interference. The drift may be due to the alternating electrode-serosa impedance, as well as body movements and possibly the electrical or mechanical activity of the heart or both. Low pass filtering and resampling are commonly used to isolate the EGG frequency spectra. While some amount of post-processing is unavoidable, limiting the extent to which signal frequencies are selected during analysis is always preferable. To reduce high frequency noise and improve the signal-noise ratio, we used a single moving average filter with a small window size of 0.006 s on both strain gauge and EGG recordings. The small window size was chosen to minimize excessive smoothing. No additional filtering or resampling was performed on the data. The dominant frequency within the strain gauge and EGG signal interval were then determined by employing the Fast-Fourier Transform (FFT) as an initial step. Indeed, the FFT is not suitable for the analysis of non-stationary signals since no information can be drawn about the frequency-time relationship. Abnormalities of slow-wave initiation and conduction occur in gastroparesis, often at normal frequency. These could be missed by tools that do not capture spatial features or amplitude of the activity, among other changes that could contribute to the pathogenesis of gastroparesis. Adaptations like the short-term Fourier Transform (STFT) are also not ideal for gastric analysis due to the tradeoff between temporal and spectral resolution. A longer window loses time resolution meanwhile a shorter window conversely results in poor frequency resolution. A better technique is to utilize wavelet transforms, particularly the continuous wavelet transform (CWT), which generates information about the frequency and amplitude components as a function of time. It allows the use of longer time windows when there is a need for precise low-frequency information and shorter windows to analyze the higher frequency information. We therefore implemented a framework that processes EGG and strain gauge data to compute the continuous wavelet transform. Data was analyzed in 30-minute intervals to gain more insightful information and allow continuous observation. To speed up the FFT calculations used in CWT, given the properties of circular convolution, the time series was zero-padded to the next highest power of 2. However, a drawback to zero-padding is the introduction of edge effects, where the amplitude near the edges of the CWT is decreased due to the introduction of zeros. As a result, a cone of influence (COI) was used to demarcate the regions where edge effects can play a role. Within the COI region, it is unclear if the decrease in power is due to a true decrease in variance of the underlying signal or is an artifact of padding and so no analysis was conducted in this region. The results of CWT analysis are dependent on the choice of mother wavelet. The Morlet wavelet was used as the mother wavelet for analysis of both strain gauge and EGG recordings due to similarities with commonly known properties of EGG, such as the characteristic frequency of 5 cpm in rats. The Morlet wavelet is also very similar to the EGG response in the time-domain. To establish significance, a 95% confidence interval contour line was plotted. This was done by deriving the theoretical red noise spectrum and Gaussian white noise spectrum using equation 16-20 provided by Torrence et. al. (“A Practical Guide to Wavelet Analysis,” Bulletin of the American Meteorological Society. 1998;79 (1): 18). For deriving red-noise spectra, a lag-1 autocorrelation coefficient of 1 was assumed for gastric data. A reasonable null hypothesis can then be defined as: It is assumed that the time series has a mean spectrum, possibly given by equation (16) in Torrence et. al. (“A Practical Guide to Wavelet Analysis,” Bulletin of the American Meteorological Society. 1998;79 (1): 18); if a peak in the wavelet spectrum is significantly above this background spectrum, then it can be assumed to be a true feature with 95% confidence. Therefore, frequencies above the 95% confidence line in the spectrum are significant. To calculate the 95% confidence level, the background spectrum was multiplied with the 95th percentile value for chi-square with two degrees of freedom.

### 2.6 Statistics

All data are given as mean: standard error of mean (SEM). The significance of difference between the EGG data during both phases from the acute experiments was determined with a one-tailed unpaired t-test. p<0.05 was considered statistically significant.

### 3. Results

### 3.1 Acute

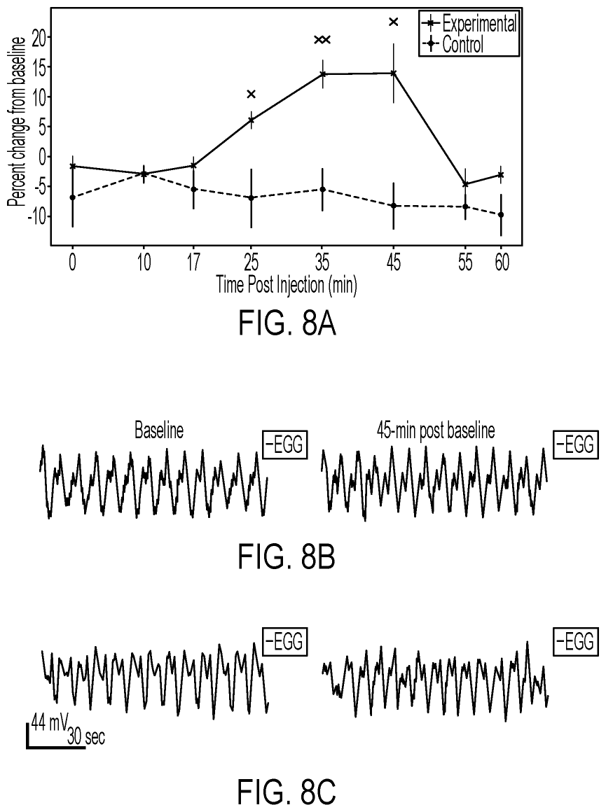

Gastric myoelectric activity recorded with EGG and gastric contraction activity recorded with the strain gauge were clearly visible across experiments. In acute experiments, FFT analysis of EGG recordings showed an average peak at 0.076±0.002 Hz, corresponding to 4.566 L 0.120 cpm, across all animals in both control and experimental groups at baseline. We analyzed the frequency of slow waves between control and experiment groups as a percent change in cycles per minute from initial baseline recording versus time post metoclopramide injection (FIG. 8A). We observed an increase in slow wave frequency following the injection, with statistically significant differences 25 mins (p=0.030768), 35 mins (p=0.00563), 45 mins (p=0.012076) post injection, with a peak change of 15% occurring 45 minutes post injection, followed by a return to near physiological baseline. In the control group, there was no statistically significant change in slow wave frequency from baseline value. The raw EGG signal from a representative animal from the control group is shown in FIG. 8B, and from a representative animal from the experimental group is shown in FIG. 8C

### 3.2 Chronic

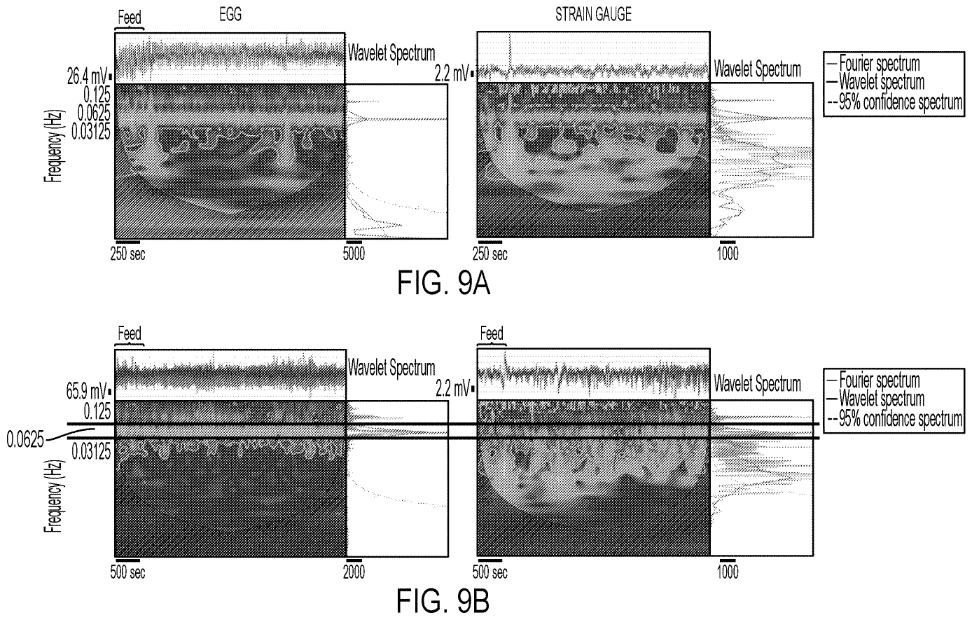

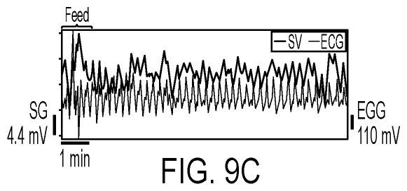

In the conscious, freely behaving state, both EGG and strain gauge activity were recorded reliably. FIG. 9A shows baseline EGG and strain gauge activity for 30 minutes preprandial, after fasting the animal for 18 h. The EGG frequency remained relatively unchanged, with a dominant frequency at 0.0625 Hz or 3.75 cpm. There is a second peak frequency identifiable at 0.125 Hz corresponding to 7.5 cpm. Strain gauge amplitude and frequency were constant, at 0.0625 Hz or 3.75 cpm. Wavelet spectral and overall signal FFT show four peaks, with components at 0.125 Hz, 0.0625 Hz, 0.0156 Hz and 0.0078 Hz. FIG. 9B shows postprandial EGG and strain gauge activity, immediately after the feeding phase, for 75 minutes. The EGG frequency decreases from an initial 4 cpm to 3 cpm, then increases to 3.5 cpm 40 minutes into the postprandial phase, lasting 17 minutes during which the amplitude increases. Wavelet spectral and overall signal FFT show two peaks, with a dominant peak at 0.0625 Hz and at 0.115 Hz. Strain gauge amplitude increases at a similar point, about 40 minutes into the postprandial phase, and was sustained up until data collection ended. Four component frequencies in the strain gauge activity were identifiable, at 0.115 Hz, 0.0625 Hz, 0.03125 Hz, and at 0.0078 Hz. FIG. 9C shows an 8-minute comparison of postprandial raw strain gauge and EGG activity, immediately following the feeding phase. The patterns shown in FIG. 7 were representative for animals in the chronic group.

### 4. Discussion

To the best of our knowledge, we believe we have developed the first fully implantable system capable of recording gastric myoelectric and contraction activity, as indications of overall gastric motility state, in freely behaving animals. Previous attempts at chronic recordings have involved tethered or exteriorized systems, not suitable for implant in humans. The results presented in the previous section demonstrate reliable recordings as well as an analysis framework that should prove very useful for understanding gastric recordings now and in the future. The acute experiments illustrate the Gastronode's ability to reliably record the expected increase in gastric myoelectric frequency after administration of an FDA approved pro-motility agent and the subsequent return to baseline. The prokinetic metoclopramide is commonly used and produces both increased gastric emptying and inhibition of nausea. However, its mechanisms of action are complex, its long-term side effects limit its use, and our data suggests it acts to increase the frequency of gastric slow waves (FIG. 8A), which is unphysiological and may have unpredictable results in some patients. The chronic experiments illustrate the Gastronode's ability to reliably record both gastric myoelectric and contraction activity in freely behaving animals at rest, as well as before and after voluntary consumption of a test meal. Naturalistic food ingestion is preferable over techniques like oral gavage or gastric inflation for a variety of reasons, including activation of vagovagal reflexes, interactions to properly activate cognitive and sensory processing in the brain, and limiting the effects of stress on gastric motility. The use of CWT can allow broader and more accurate identification of the relative proportion of time over which a particular electrophysiological frequency occurs, in addition to distinguishing patterns and changes in gastric motility state, which cannot be reliably quantified with FFT analysis. In our chronic fasting experiments, immediately after the test meal, a dip in postprandial EGG frequency was observed in every animal. The average frequency decrease was 20:4.2% of the fasting frequency. After about five minutes the frequency started to increase until it stabilized at a level which was at least equal to or slightly above the fasting frequency. This is similar to patterns observed in a previous human study. In FIG. 9B, gastric antral contraction frequency remains relatively unchanged. The interstitial cells of Cajal set the basal electrical rhythm of contractions. We expected to see more amplitude changes in antral contractions, rather than frequency, as evidenced in FIG. 9B, and in line with previous reported studies.

While the clinical distinctions between different types of gastric motor disorders are complex, patients with the constellation of symptoms related to gastric dysfunction ranging from postprandial discomfort and premature satiety to bloating, nausea, vomiting or regurgitation and epigastric pain are generally divided into 2 groups based on measurement of gastric emptying, with patients showing delayed emptying classed as having gastroparesis while those without are classed as having functional dyspepsia. Recent work by Pasrisha et al. (“Functional Dyspepsia and Gastroparesis in Tertiary Care are Interchangeable Syndromes With Common Clinical and Pathologic Features,” Gastroenterology. 2021; 160 (6): 2006-2017), however, suggests that a single measure of gastric emptying, with low temporal resolution is not a reliable means of defining the underlying pathology causing symptoms in this broader patient group. Patients with these symptoms can also be divided into 2 main groups based on the etiology of disease, that is diabetic and idiopathic. Both etiological groups showed a lack of test/retest reliability with respect to their classification as having gastroparesis or FD. The study authors concluded that there may not be a real difference between these 2 disorders of gastric motility and sensation. Together, this combined cohort represents a very large group of patients for whom their symptoms are debilitating and the therapies available to them are limited. As mentioned prior, despite the heterogeneity of gastric motility observed in patients, a unifying feature is a lack of coordination between contractions and electrical activity in different gastric regions and/or between antral contractions, pyloric opening. A tool like the Gastronode can solve this unmet need and allow continuous recording of contraction and electrical activity in freely behaving subjects.

### 5. Conclusion

The authors conclude that the device developed in this work can allow measurement of gastric contractive and electrical slow wave activity, enabling further studies into future closed loop stimulation therapies that can be delivered with physiologically appropriate timing and extent. A device developed and validated to record gastric activity in freely behaving animals at rest, and before and after food intake can enable chronic studies into the efficacy of GES and inspire the development of novel therapies that can improve clinical outcomes.

Some further aspects are defined in the following clauses:
Clause 1: An implantable electroceutical device to treat and monitor a gastric disorder in a subject, comprising: a gastric motility sub-assembly configured to record gastric myoelectric activity and gastric contraction activity in the subject when the implantable electroceutical device is operably implanted in the subject; a gastric electrical stimulation (GES) sub-assembly configured to electrically stimulate a nervous system of the subject when the implantable electroceutical device is operably implanted in the subject; a controller operably connected, or connectable, to the gastric motility sub-assembly and to the GES sub-assembly, which controller comprises, or is capable of accessing, computer readable media comprising non-transitory computer-executable instructions, which when executed by at least one electronic processor, perform at least: effecting recording of the gastric myoelectric activity and the gastric contraction activity in the subject using the gastric motility sub-assembly when the implantable electroceutical device is operably implanted in the subject; and effecting electrically stimulating the nervous system of the subject using the GES sub-assembly when the implantable electroceutical device is operably implanted in the subject; and, a power source operably connected, or connectable, to the controller, the gastric motility sub-assembly, and/or the GES sub-assembly.
Clause 2: The implantable electroceutical device of Clause 1, wherein the gastric motility sub-assembly comprises an electrogastrography (EEG) apparatus configured to record the gastric myoelectric activity in the subject when the implantable electroceutical device is operably implanted in the subject.
Clause 3: The implantable electroceutical device of Clause 1 or Clause 2, wherein the gastric motility sub-assembly comprises a strain gauge force transducer configured to record the gastric contraction activity in the subject when the implantable electroceutical device is operably implanted in the subject.
Clause 4: The implantable electroceutical device of any one of the preceding Clauses 1-3, wherein the gastric motility sub-assembly configured to record physiologically relevant gastric motility patterns and changes in the subject when the implantable electroceutical device is operably implanted in the subject.
Clause 5: The implantable electroceutical device of any one of the preceding Clauses 1-4, wherein the gastric motility sub-assembly is configured to substantially continuously record the gastric myoelectric activity and the gastric contraction activity in the subject when the implantable electroceutical device is operably implanted in the subject.
Clause 6: The implantable electroceutical device of any one of the preceding Clauses 1-5, wherein the non-transitory computer-executable instructions, when executed by the electronic processor, are configured to effect recording of the gastric myoelectric activity and the gastric contraction activity in the subject and to effect electrically stimulating the nervous system of the subject substantially synchronous with one another.
Clause 7: The implantable electroceutical device of any one of the preceding Clauses 1-6, wherein the non-transitory computer-executable instructions, when executed by the electronic processor, are configured to use continuous wavelet transforms to analyze frequency and amplitude changes in captured data to inform potential therapies for the subject when the implantable electroceutical device is operably implanted in the subject.
Clause 8: The implantable electroceutical device of any one of the preceding Clauses 1-7, wherein the non-transitory computer-executable instructions, when executed by the electronic processor, are configured to effect selective stimulation of enteric neurons of the subject in synchronization with recording gastric slow waves in the subject when the implantable electroceutical device is operably implanted in the subject.
Clause 9: The implantable electroceutical device of any one of the preceding Clauses 1-8, wherein the controller is configured for bi-directional wireless communication with a data acquisition computer readable media that is external to the subject.
Clause 10: The implantable electroceutical device of any one of the preceding Clauses 1-9, wherein the gastric disorder comprises gastroparesis and/or functional dyspepsia (FD).
Clause 11: The implantable electroceutical device of any one of the preceding Clauses 1-10, wherein the implantable electroceutical device is configured for chronic implantation in the subject.
Clause 12: The implantable electroceutical device of any one of the preceding Clauses 1-11, wherein the implantable electroceutical device is configured to administer closed-loop therapy to the subject when the implantable electroceutical device is operably implanted in the subject.
Clause 13: The implantable electroceutical device of any one of the preceding Clauses 1-12, wherein the closed-loop therapy increases gastric accommodation, stimulates physiological gastric emptying, and/or stimulates pyloric opening in the subject when the implantable electroceutical device is operably implanted in the subject.
Clause 14: The implantable electroceutical device of any one of the preceding Clauses 1-13, further comprising at least one external drive coil, at least one external base station, and/or at least one external computer operably connected, or connectable, to the implantable electroceutical device when the implantable electroceutical device is operably implanted in the subject.
Clause 15: The implantable electroceutical device of any one of the preceding Clauses 1-14, further comprising one or more electrodes positioned within sensory communication of a stomach of the subject and operably connected to the implantable electroceutical device when the implantable electroceutical device is operably implanted in the subject.
Clause 16: A system comprising the implantable electroceutical device of any one of the preceding Clauses 1-15.
Clause 17: A kit comprising the implantable electroceutical device of any one of the preceding Clauses 1-15.
Clause 18: A method of treating and monitoring a gastric disorder in a subject, the method comprising: recording gastric myoelectric activity and gastric contraction activity in the subject having the gastric disorder using an implantable electroceutical device that is operably implanted in the subject; and, electrically stimulating a nervous system of the subject having the gastric disorder using the implantable electroceutical device that is operably implanted in the subject, thereby treating and monitoring the gastric disorder in the subject.
Clause 19: The method of Clause 18, further comprising operably implanting the implantable electroceutical device in the subject.
Clause 20: The method of Clause 18 or Clause 19, wherein the implantable electroceutical device comprises: a gastric motility sub-assembly configured to record gastric myoelectric activity and gastric contraction activity in the subject; a gastric electrical stimulation (GES) sub-assembly configured to electrically stimulate the nervous system of the subject; a controller operably connected, or connectable, to the gastric motility sub-assembly and to the GES sub-assembly, which controller comprises, or is capable of accessing, computer readable media comprising non-transitory computer-executable instructions, which when executed by at least one electronic processor, perform at least: effecting the recording of the gastric myoelectric activity and the gastric contraction activity in the subject using the gastric motility sub-assembly; and effecting the electrically stimulating of the nervous system of the subject using the GES sub-assembly; and, a power source operably connected, or connectable, to the controller, the gastric motility sub-assembly, and/or the GES sub-assembly.
Clause 21: A computer readable media comprising non-transitory computer-executable instructions which, when executed by at least one electronic processor perform at least: effecting recording of gastric myoelectric activity and gastric contraction activity in a subject using a gastric motility sub-assembly of an implantable electroceutical device that is operably implanted in the subject; and, effecting electrically stimulating a nervous system of the subject using a gastric electrical stimulation (GES) sub-assembly of the implantable electroceutical device that is operably implanted in the subject.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. An implantable electroceutical device to treat and monitor a gastric disorder in a subject, comprising:
a gastric motility sub-assembly configured to record gastric myoelectric activity and gastric contraction activity in the subject when the implantable electroceutical device is operably implanted in the subject;
a gastric electrical stimulation (GES) sub-assembly configured to electrically stimulate a nervous system of the subject when the implantable electroceutical device is operably implanted in the subject;
a controller operably connected, or connectable, to the gastric motility sub-assembly and to the GES sub-assembly, which controller comprises, or is capable of accessing, computer readable media comprising non-transitory computer-executable instructions, which when executed by at least one electronic processor, perform at least:
effecting recording of the gastric myoelectric activity and the gastric contraction activity in the subject using the gastric motility sub-assembly when the implantable electroceutical device is operably implanted in the subject; and
effecting electrically stimulating the nervous system of the subject using the GES sub-assembly when the implantable electroceutical device is operably implanted in the subject; and,
a power source operably connected, or connectable, to the controller, the gastric motility sub-assembly, and/or the GES sub-assembly.

2. The implantable electroceutical device of claim 1, wherein the gastric motility sub-assembly comprises an electrogastrography (EEG) apparatus configured to record the gastric myoelectric activity in the subject when the implantable electroceutical device is operably implanted in the subject.

3. The implantable electroceutical device of claim 1, wherein the gastric motility sub-assembly comprises a strain gauge force transducer configured to record the gastric contraction activity in the subject when the implantable electroceutical device is operably implanted in the subject.

4. The implantable electroceutical device of claim 1, wherein the gastric motility sub-assembly configured to record physiologically relevant gastric motility patterns and changes in the subject when the implantable electroceutical device is operably implanted in the subject.

5. The implantable electroceutical device of claim 1, wherein the gastric motility sub-assembly is configured to substantially continuously record the gastric myoelectric activity and the gastric contraction activity in the subject when the implantable electroceutical device is operably implanted in the subject.

6. The implantable electroceutical device of claim 1, wherein the non-transitory computer-executable instructions, when executed by the electronic processor, are configured to effect recording of the gastric myoelectric activity and the gastric contraction activity in the subject and to effect electrically stimulating the nervous system of the subject substantially synchronous with one another.

7. The implantable electroceutical device of claim 1, wherein the non-transitory computer-executable instructions, when executed by the electronic processor, are configured to use continuous wavelet transforms to analyze frequency and amplitude changes in captured data to inform potential therapies for the subject when the implantable electroceutical device is operably implanted in the subject.

8. The implantable electroceutical device of claim 1, wherein the non-transitory computer-executable instructions, when executed by the electronic processor, are configured to effect selective stimulation of enteric neurons of the subject in synchronization with recording gastric slow waves in the subject when the implantable electroceutical device is operably implanted in the subject.

9. The implantable electroceutical device of claim 1, wherein the controller is configured for bi-directional wireless communication with a data acquisition computer readable media that is external to the subject.

10. The implantable electroceutical device of claim 1, wherein the gastric disorder comprises gastroparesis and/or functional dyspepsia (FD).

11. The implantable electroceutical device of claim 1, wherein the implantable electroceutical device is configured for chronic implantation in the subject.

12. The implantable electroceutical device of claim 1, wherein the implantable electroceutical device is configured to administer closed-loop therapy to the subject when the implantable electroceutical device is operably implanted in the subject.

13. The implantable electroceutical device of claim 1, wherein the closed-loop therapy increases gastric accommodation, stimulates physiological gastric emptying, and/or stimulates pyloric opening in the subject when the implantable electroceutical device is operably implanted in the subject.

14. The implantable electroceutical device of claim 1, further comprising at least one external drive coil, at least one external base station, and/or at least one external computer operably connected, or connectable, to the implantable electroceutical device when the implantable electroceutical device is operably implanted in the subject.

15. The implantable electroceutical device of claim 1, further comprising one or more electrodes positioned within sensory communication of a stomach of the subject and operably connected to the implantable electroceutical device when the implantable electroceutical device is operably implanted in the subject.

16. A system comprising the implantable electroceutical device of claim 1.

17. A kit comprising the implantable electroceutical device of claim 1.

18. A method of treating and monitoring a gastric disorder in a subject, the method comprising:
recording gastric myoelectric activity and gastric contraction activity in the subject having the gastric disorder using an implantable electroceutical device that is operably implanted in the subject; and,
electrically stimulating a nervous system of the subject having the gastric disorder using the implantable electroceutical device that is operably implanted in the subject, thereby treating and monitoring the gastric disorder in the subject.

19. (canceled)

20. The method of any one of claim 18, wherein the implantable electroceutical device comprises:
a gastric motility sub-assembly configured to record gastric myoelectric activity and gastric contraction activity in the subject;
a gastric electrical stimulation (GES) sub-assembly configured to electrically stimulate the nervous system of the subject;
a controller operably connected, or connectable, to the gastric motility sub-assembly and to the GES sub-assembly, which controller comprises, or is capable of accessing, computer readable media comprising non-transitory computer-executable instructions, which when executed by at least one electronic processor, perform at least:
effecting the recording of the gastric myoelectric activity and the gastric contraction activity in the subject using the gastric motility sub-assembly; and
effecting the electrically stimulating of the nervous system of the subject using the GES sub-assembly; and,
a power source operably connected, or connectable, to the controller, the gastric motility sub-assembly, and/or the GES sub-assembly.

21. A computer readable media comprising non-transitory computer-executable instructions which, when executed by at least one electronic processor perform at least:
effecting recording of gastric myoelectric activity and gastric contraction activity in a subject using a gastric motility sub-assembly of an implantable electroceutical device that is operably implanted in the subject; and,
effecting electrically stimulating a nervous system of the subject using a gastric electrical stimulation (GES) sub-assembly of the implantable electroceutical device that is operably implanted in the subject.
